(19) United States
(12) Patent Application Publication
(10) Pub. No.: US 2022/0333067 A1
(43) Pub. Date: Oct. 20, 2022

(54) PLURIPOTENT STEM CELL MANUFACTURING SYSTEM

(71) Applicant: Canon Medical Systems Corporation, Otawara-shi (JP)

(72) Inventors: Kei MORI, Brookline, MA (US); Kohei WATANABE, Utsunomiya (JP)

(73) Assignee: Canon Medical Systems Corporation, Otawara-shi (JP)

(21) Appl. No.: 17/658,284

(22) Filed: Apr. 7, 2022

(30) Foreign Application Priority Data
Apr. 19, 2021 (JP) 2021-070452

(51) Int. Cl. C12N 5/074; C12N 5/00; C12N 5/0735; C12N 5/079; C12N 13/00

(52) U.S. Cl. CPC C12N 5/0607; C12N 5/0081; C12N 5/0606; C12N 5/0622; C12N 13/00; C12N 2502/09; C12N 2502/23; C12N 2506/02

(57) ABSTRACT

According to one embodiment, a pluripotent stem cell manufacturing system includes processing circuitry. The processing circuitry acquires storage information for the storage of a sample from a donor, and surviving cell number information for the number of surviving cells contained in the sample. The processing circuitry estimates tissue stem cell number information for the number of tissue stem cells contained in the sample, based on the storage information and the surviving cell number information.

### CROSS-REFERENCE TO RELATED APPLICATIONS

This application is based upon and claims the benefit of priority from Japanese Patent Application No. 2021-070452, filed Apr. 19, 2021, the entire contents of which are incorporated herein by reference.

### FIELD

Embodiments described herein relate generally to a pluripotent stem cell manufacturing system.

### BACKGROUND

Development of regenerative medicine has seen proposals for various pluripotent stem cell manufacturing systems. There is a need for manufacturing pluripotent stem cells from donor's own blood, but reduced costs and increased throughput are concurrently desired. Efficient manufacturing of pluripotent stem cells according to the conditions of blood used is also desired. While simplicity and convenience in manufacturing processes should be pursued, failure in manufacturing is not tolerated, and as such, a manufacturing mechanism that satisfies both of such aspects is sought.

### BRIEF DESCRIPTION OF THE DRAWINGS

FIG. 1 is a diagram showing an exemplary configuration of a pluripotent stem cell manufacturing system according to an embodiment.

FIG. 2 is a diagram showing an exemplary configuration of a tissue stem cell number estimating apparatus shown in FIG. 1.

FIG. 3 is a diagram showing an exemplary configuration of a pluripotent stem cell manufacturing apparatus shown in FIG. 1.

FIG. 4 is a diagram showing an exemplary operation of manufacturing induced pluripotent stem cells by the pluripotent stem cell manufacturing system shown in FIG. 1.

FIG. 5 is a diagram schematically showing input and output of a trained model used in step S3 shown in FIG. 4.

FIG. 6 is a diagram showing an example of a manufacturing process table used in step S4 shown in FIG. 4.

FIG. 7 is a diagram showing an example of a display screen displaying, in step S5 shown in FIG. 4, hematopoietic stem cell number information and a manufacturing process.

FIG. 8 is a diagram showing another example of a display screen displaying, in step S5 shown in FIG. 4, hematopoietic stem cell number information and a manufacturing process.

FIG. 9 is a diagram schematically showing a process for manufacturing induced pluripotent stem cells in the case of level “1” (extended cultivation omitted).

FIG. 10 is a diagram schematically showing a process for manufacturing induced pluripotent stem cells in the case of level “2” (short cultivation).

FIG. 11 is a diagram schematically showing a process for manufacturing induced pluripotent stem cells in the case of level “3” (regular cultivation).

FIG. 12 is a diagram showing another example of a display screen presented in step S5 shown in FIG. 4.

FIG. 13 is a diagram schematically showing a training process for the trained model and the manufacturing process table.

### DETAILED DESCRIPTION

According to an embodiment, a pluripotent stem cell manufacturing system includes processing circuitry. The processing circuitry acquires storage information for the storage of a sample from a donor, and surviving cell number information for the number of surviving cells contained in the sample. The processing circuitry estimates tissue stem cell number information for the number of tissue stem cells contained in the sample, based on the storage information and the surviving cell number information.

Embodiments of a pluripotent stem cell manufacturing system will be described in detail with reference to the drawings.

A pluripotent stem cell manufacturing system according to an embodiment is, for example, a mechanical system adapted to manufacture pluripotent stem cells having an origin in a donor, through the use of tissue stem cells contained in a sample taken from the donor. The pluripotent stem cells in the context of the embodiment refer to cells with pluripotency, such as embryonic stem cells (ES cells), nuclear transfer embryonic stem cells (ntES cells), and induced pluripotent stem cells (iPS cells). Note that “induced” in the term “induced pluripotent stem cells” may be understood as “artificially induced”. The tissue stem cells refer to cells with multilineage differentiation potential, such as hematopoietic stem cells, neural stem cells, hepatic stem cells, renal stem cells, and skin stem cells. The sample may contain any tissue from blood, bone marrow, skin, etc. of the donor. The donor may be any subject, such as a human being, an animal, etc.

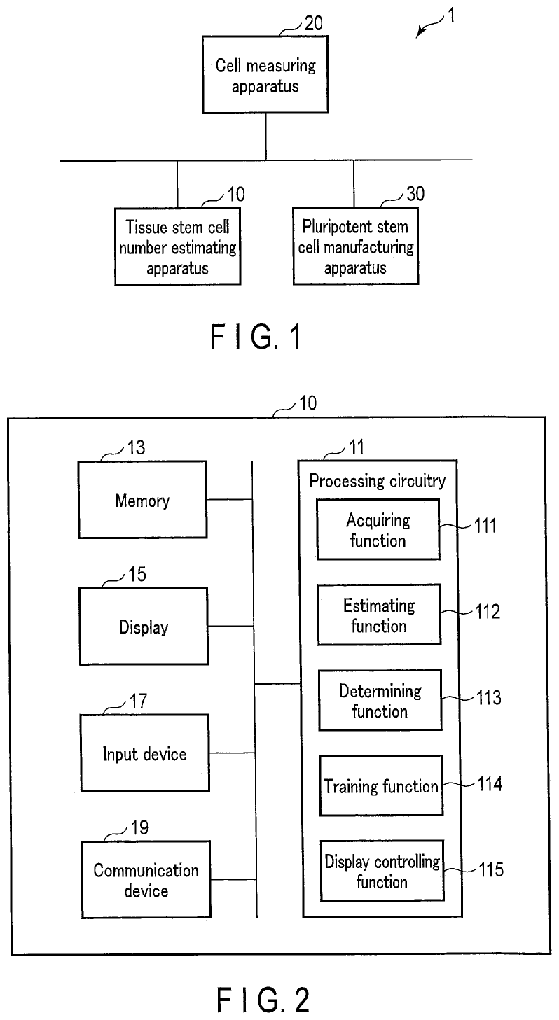

FIG. 1 is a diagram showing an exemplary configuration of a pluripotent stem cell manufacturing system 1 according to the embodiment. The pluripotent stem cell manufacturing system 1 includes a tissue stem cell number estimating apparatus 10, a cell measuring apparatus 20, and a pluripotent stem cell manufacturing apparatus 30.

The tissue stem cell number estimating apparatus 10 is, for example, a computer for estimating information for the number of tissue stem cells contained in a sample from the donor (this information will be called “tissue stem cell number information”). The cell measuring apparatus 20 is, for example, a mechanical device utilizing various principles for measuring cells contained in the sample from the donor. The pluripotent stem cell manufacturing apparatus 30 is, for example, a mechanical system for manufacturing pluripotent stem cells having an origin in the donor, through the use of the sample from the donor. The pluripotent stem cell manufacturing apparatus 30 manufactures the pluripotent stem cells according to a manufacturing process that suits the tissue stem cell number information estimated by the tissue stem cell number estimating apparatus 10.

FIG. 2 is a diagram showing an exemplary configuration of the tissue stem cell number estimating apparatus 10 shown in FIG. 1. As shown in FIG. 2, the tissue stem cell number estimating apparatus 10 includes processing circuitry 11, a memory 13, a display 15, an input device 17, and a communication device 19. The processing circuitry 11, the memory 13, the display 15, the input device 17, and the communication device 19 are connected with one another via a bus so that they can communicate with one another.

The processing circuitry 11 includes one or more processors. Such one or more processors execute one or more programs for the embodiment so as to realize at least one of an acquiring function 111, an estimating function 112, a determining function 113, a training function 114, and/or a display controlling function 115. The one or more programs are stored in one or more computer readable recording media such as the memory 13, a portable recording medium, etc.

By implementing the acquiring function 111, the processing circuitry 11 acquires various information sets. For example, the processing circuitry 11 acquires information for the storage of a sample from the donor (this information will be called “storage information”). The processing circuitry 11 also acquires information for the number of surviving cells contained in the sample from the donor (this information will be called “surviving cell number information”). A sample or samples are stored in advance in any given depository or depositories. The sample for use is provided from the depository to the pluripotent stem cell manufacturing system 1. The storage information is about the storage of this sample in the depository. The surviving cell number information is about the number of surviving cells contained in the sample after the storage in the depository and before the start of processing by the pluripotent stem cell manufacturing apparatus 30. The sample may contain not only the tissue stem cells for use in the manufacturing of pluripotent stem cells but also many kinds of cells which may be handled as unwanted substances subject to removal. The surviving cells refer to cells that are alive in the sample, and may include not only the tissue stem cells but also many kinds of cells. The surviving cell number information is a factor affecting the success or the failure of the manufacturing of pluripotent stem cells, and serves as one piece of exemplary information for the estimation of blood sample conditions or surviving cell conditions.

By implementing the estimating function 112, the processing circuitry 11 estimates information for the number of tissue stem cells contained in the sample (this information will be called “tissue stem cell number information”), based on the storage information and the surviving cell number information acquired by the acquiring function 111. The tissue stem cell number information is a factor affecting the success or the failure of the manufacturing of pluripotent stem cells, and serves as another piece of exemplary information for the estimation of blood sample conditions or surviving cell conditions. In one example, the processing circuitry 11 applies the storage information and the surviving cell number information to a trained model to estimate the tissue stem cell number information.

By implementing the determining function 113, the processing circuitry 11 determines, based on the tissue stem cell number information estimated by the estimating function 112, the manufacturing process for manufacturing pluripotent stem cells from the tissue stem cells. In one example, the processing circuitry 11 determines the manufacturing process based on a table which associates the levels for the tissue stem cell number information with manufacturing processes (this table will be called a “manufacturing process table”).

By implementing the training function 114, the processing circuitry 11 trains one or more machine learning models for use by the estimating function 112, one or more manufacturing process tables for use by the determining function 113, and so on.

By implementing the display controlling function 115, the processing circuitry 11 presents various information sets through the display 15. For example, the processing circuitry 11 presents the tissue stem cell number information estimated by the estimating function 112, the manufacturing process determined by the determining function 113, and so on.

The memory 13 is a storage device such as a RAM, a ROM, a hard disk drive (HDD), a solid state drive (SSD), or a semiconductor memory device, adapted to store various information items. For example, the memory 13 stores various programs and the like. Note that the memory 13, as a hardware device, may also be a drive unit or the like, such as a CD-ROM drive, a DVD drive, a flash memory, etc., adapted to read and write various information sets from and to portable recording media.

The display 15 displays various information items. Examples which may be discretionarily adopted as the display 15 include a CRT display, a liquid crystal display, an organic EL display, an LED display, a plasma display, or any other display known in this technical field. The display 15 may instead or additionally be a projector device.

The input device 17 is an interface for inputting various commands from an operator, etc. The input device 17 may be constituted by a keyboard, a mouse, and any type of switch, etc. The input device 17 sends output signals corresponding to the various commands to the processing circuitry 11 via the bus.

The communication device 19 performs data communication with entities such as the cell measuring apparatus 20 and the pluripotent stem cell manufacturing apparatus 30 through non-illustrated communication lines or in a wireless manner. For example, the communication device 19 receives the surviving cell number information from the cell measuring apparatus 20. Also, the communication device 19 sends information for the manufacturing process determined by the determining function 113 to the pluripotent stem cell manufacturing apparatus 30.

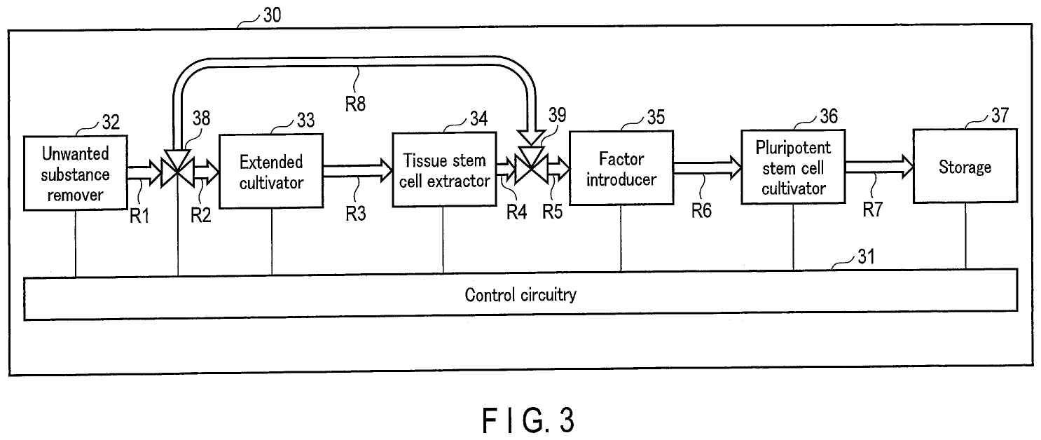

FIG. 3 is a diagram showing an exemplary configuration of the pluripotent stem cell manufacturing apparatus 30 shown in FIG. 1. As shown in FIG. 3, the pluripotent stem cell manufacturing apparatus 30 includes control circuitry 31, an unwanted substance remover 32, an extended cultivator 33, a tissue stem cell extractor 34, a factor introducer 35, a pluripotent stem cell cultivator 36, a storage 37, a first switcher 38, and a second switcher 39.

The control circuitry 31 includes, for example, a processor such as a CPU, a GPU, or the like. The control circuitry 31 controls the unwanted substance remover 32, the extended cultivator 33, the tissue stem cell extractor 34, the factor introducer 35, the pluripotent stem cell cultivator 36, the storage 37, the first switcher 38, and the second switcher 39, so that pluripotent stem cells having an origin in a donor are manufactured from the tissue stem cells contained in a blood sample according to the manufacturing process that suits the tissue stem cell number information estimated by the tissue stem cell number estimating apparatus 10.

The unwanted substance remover 32 is, for example, a mechanical device for removing one or more unwanted substances from the sample from a donor. Examples which may be adopted as the unwanted substance remover 32 include a centrifugal separator, a filtering device, etc. Removal of the unwanted substances allows for extraction of tissue stem cells. The unwanted substance remover 32 is connected to the extended cultivator 33 via a flow channel R1, the first switcher 38, and a flow channel R2. A fluid containing the tissue stem cells is fed to the extended cultivator 33 via the flow channel R1, the first switcher 38, and the flow channel R2.

The extended cultivator 33 is, for example, a mechanical device for carrying out extended cultivation of tissue stem cells. In one example, the extended cultivator 33 includes a culture vessel and a dispensing mechanism. The dispensing mechanism is connected to the flow channel R2. The dispensing mechanism aspirates the fluid containing the tissue stem cells and discharges it to the culture vessel. The culture vessel may contain various reagents including a desired culture medium, added as appropriate by the dispensing mechanism, etc. Cultivation time at the extended cultivator may be discretionarily adjusted. The extended cultivator 33 is connected to the tissue stem cell extractor 34 via a flow channel R3. A fluid containing the tissue stem cells that have undergone the extended cultivation is fed to the tissue stem cell extractor 34 via the flow channel R3. Here, since the extended cultivator 33 is also fed with a subtle amount of unwanted substances together with the tissue stem cells, the unwanted substances likewise grow with the tissue stem cells.

The tissue stem cell extractor 34 extracts the tissue stem cells from the fluid containing the tissue stem cells, fed from the extended cultivator 33. In one example, a flow cytometer device is used as the tissue stem cell extractor 34. Such a flow cytometer device is connected to the flow channel R3. The flow cytometer device aligns cells contained in the fluid from the extended cultivator 33 to then optically detect, count, and sort the cells. This accordingly realizes extraction of the tissue stem cells. The tissue stem cell extractor 34 is connected to the factor introducer 35 via a flow channel R4, the second switcher 39, and a flow channel R5.

The factor introducer 35 establishes pluripotent stem cells by introducing an inducing factor into the tissue stem cells extracted by the tissue stem cell extractor 34. In one example, the factor introducer 35 includes a flow path for the fluid containing the tissue stem cells to flow, and a pump for feeding the inducing factor to this flow path. The inducing factor here is a factor for reprogramming the tissue stem cells, and a factor known as a “Yamanaka factor” may be used. As concrete examples, one or more of Oct family genes, Klf family genes, Myc family genes, and their respective gene products may be used. In one example, Oct3/4 is used as the Oct family gene, Klf4 is used as the Klf family gene, and c-Myc or L-Myc is used as the Myc family gene. The inducing factor may also include Sox family genes and/or gene products. As the Sox family gene, Sox2 may be used. When the inducing factor is introduced into tissue stem cells, the tissue stem cells are reprogrammed so that pluripotent stem cells are established. The factor introducer 35 is connected to the pluripotent stem cell cultivator 36 via a flow channel R6. A fluid containing the pluripotent stem cells is fed to the pluripotent stem cell cultivator 36 via the flow channel R6.

The pluripotent stem cell cultivator 36 cultivates the pluripotent stem cells fed from the factor introducer 35. In one concrete example, the pluripotent stem cell cultivator 36 includes a culture vessel and a dispensing mechanism. The dispensing mechanism dispenses the fluid containing pluripotent stem cells to the culture vessel. The dispensing mechanism may add various reagents, such as a culture medium, to the culture vessel as appropriate. Using a given cultivation time, multiple clusters of pluripotent stem cells are manufactured within the culture vessel. The pluripotent stem cell cultivator 36 is connected to the storage 37 via a flow channel R7. The cell clusters are fed to the storage 37 via the flow channel R7.

The storage 37 stores the pluripotent stem cells fed from the pluripotent stem cell cultivator 36. In one concrete example, the storage 37 freezes the clusters of the pluripotent stem cells with a cryopreservative solution and seal or encapsulate them in one or more desired containers.

As shown in FIG. 3, the unwanted substance remover 32 is selectively connected to the extended cultivator 33 and the factor introducer 35 via the first switcher 38. More specifically, the unwanted substance remover 32 and the first switcher 38 are connected to each other via the flow channel R1, and the first switcher 38 and the extended cultivator 33 are connected to each other via the flow channel R2. Also, the second switcher 39 is arranged between the tissue stem cell extractor 34 and the factor introducer 35. The tissue stem cell extractor 34 and the second switcher 39 are connected to each other via the flow channel R4, and the second switcher 39 and the factor introducer 35 are connected to each other via the flow channel R5. The first switcher 38 and the second switcher 39 are connected to each other via a flow channel R8.

The first switcher 38 is constituted by, for example, a three-way valve capable of opening and closing individual ones of the outlet to the flow channel R2 (hereinafter, a “first outlet”) and the outlet to the flow channel R8 (hereinafter, a “second outlet”). The first switcher 38 individually opens or closes the first outlet and the second outlet according to one or more switch signals from the control circuitry 31.

The second switcher 39 is constituted by, for example, a three-way valve capable of opening and closing individual ones of the inlet from the flow channel R4 (hereinafter, a “first inlet”) and the inlet from the flow channel R8 (hereinafter, a “second inlet”). The second switcher 39 individually opens or closes the first inlet and the second inlet according to one or more switch signals from the control circuitry 31.

Next, exemplary operations of the pluripotent stem cell manufacturing system 1 having the above configuration will be described. Note that the description will assume, by way of example, the donor to be a fetus, the sample to be a blood sample, the tissue stem cells to be hematopoietic stem cells, and the pluripotent stem cells to be induced pluripotent stem cells (iPS cells). The blood sample is not limited to a particular type, but may be peripheral blood, umbilical blood, etc. Peripheral blood mononuclear cells (PBMC) fractionated from the blood of any part of the donor may also be used. However, by way of example, the description will assume the blood sample to be umbilical blood.

Umbilical blood here refers to fetal blood contained in the umbilical cord connecting a fetus and a mother. Umbilical blood contains undifferentiated cell fractions in CD34+ cells, namely, CD34+CD33-cells and CD34+CD38-cells at high proportions. That is, umbilical blood contains an abundance of highly undifferentiated hematopoietic stem cells. As such, umbilical blood has been recognized as a suitable material for the manufacturing of induced pluripotent stem cells. Note that peripheral blood of an adult contains almost no hematopoietic stem cells. Umbilical blood banks have been instituted for the transplantation of hematopoietic stem cells, and approximately 10,000 samples each having a collected quantity of 40 to 150 ml are stored in a frozen state in Japan. As these samples are to be used for transplantation, guidelines for the quality assurance standard have been set and the samples are stored appropriately. In other words, the samples have been checked under strict standards at the initial time point for their storage. Meanwhile, the survival rate of CD34+ cells after thawing is in the range of 34% to 99% depending on the circumstances in each blood bank, such as a storage period and storage conditions, and it has been reported that umbilical blood samples vary in their conditions.

Manufacture of iPS cells from autologous blood is being attempted. As the autologous blood, use of various types of blood as mentioned above is being studied. For iPS cells from autologous blood, a robust manufacturing method that can deal with various blood samples in various conditions is desired. Simplicity and convenience in manufacturing processes should be pursued in order to attain improved throughput and low cost at the same time. However, failure in manufacturing is not tolerated, and therefore, a manufacturing mechanism that satisfies both of such aspects is sought.

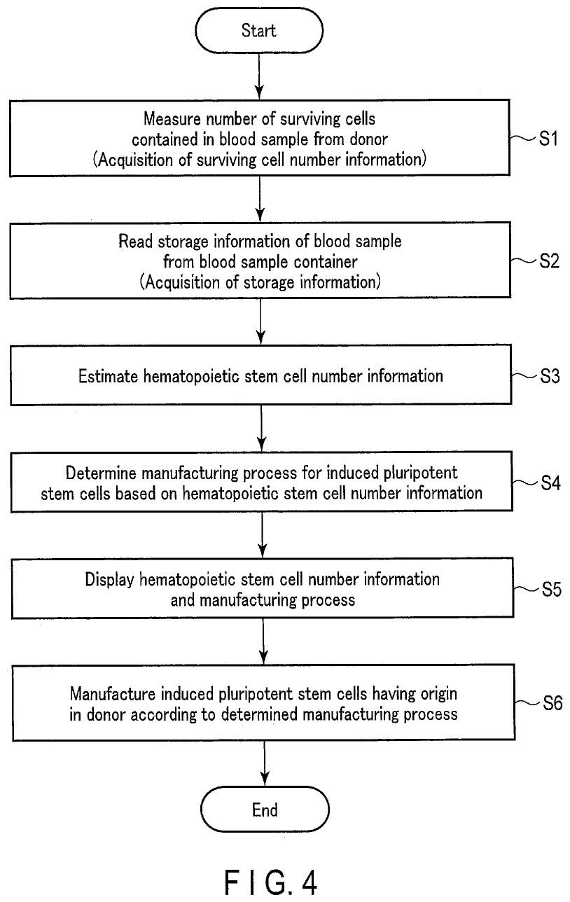

FIG. 4 is a diagram showing an exemplary operation of manufacturing induced pluripotent stem cells by the pluripotent stem cell manufacturing system 1 shown in FIG. 1. As shown in FIG. 4, the cell measuring apparatus 20 measures the number of surviving cells contained in a blood sample (input blood) from the donor (step S1). The blood sample is, for example, stored in a frozen state in a given umbilical blood depository such as an umbilical blood bank. The blood sample is thawed and then used for manufacturing pluripotent stem cells. In the course of freezing, storing, and thawing of the blood sample, some cells in the blood sample die. After the thawing of the blood sample, the cell measuring apparatus 20 measures the number of surviving cells contained in the thawed blood sample. Preferably, the amount of each blood sample undergoing the measurement is standardized to an arbitrary equal amount, e.g., 10 ml or any discretionarily set amount.

However, no limitation is intended by this.

The cell measuring apparatus 20 may be, for example, a cell counter. In this instance, the cell measuring apparatus 20 generates one or more optical images by imaging the blood sample using an optical imaging device, and subjects such one or more optical images to image processing so as to measure the number or the ratio of surviving cells. The cell measuring apparatus 20 may instead or additionally generate one or more optical spectra by optically detecting the blood sample, and analyze such one or more optical spectra so as to measure the number or the ratio of surviving cells. The ratio of surviving cells here may be defined to be a ratio of the number of surviving cells to the number of all the cells contained in the blood sample. Numerical information for the number or the ratio of surviving cells is one example of the surviving cell number information. The surviving cell number information serves as information for the estimation of the current conditions of the blood sample. The surviving cell number information is acquired by the processing circuitry 11 with the acquiring function 111.

After step S1, the processing circuitry 11 also implements the acquiring function 111 to read the storage information of the blood sample from the blood sample container (step S2). The storage information is information for the storage of the blood sample, which also enables the estimation of the current conditions of the blood sample. The blood sample container is affixed with a blood data sheet. The blood data sheet may carry a printed or attached code which is a one-dimensional or two-dimensional code or the like associated with the storage information including, for example, storage conditions in the depository for the blood sample. For example, blood depositories such as an umbilical blood bank keep records of storage conditions for each blood sample, which include the numbers of blood cells and CD34 positive cells at the initial time point for the storage, the number or ratio of the CD34 positive cells, the number of storage years, the storage period, and so on. The processing circuitry 11 acquires the storage information associated with such a printed or attached code on the blood sample container, by reading the code using a code reader. The blood data sheet may instead or additionally carry printed character, numerals, etc., representing the storage information. Also in such instances, the processing circuitry 11 may use an optical character reader, etc., to read the character, numerals, or the like printed on the blood data sheet so as to acquire the storage information represented by the character, numerals, or the like.

The acquired storage information may include identification information of the donor. The identification information of the donor may be constituted by the name, age, weight, race, etc. of the donor or any information affecting the quality of the blood sample. A code or codes for the identification information of the donor may also be printed on the blood data sheet. Characters, numerals, etc., representing the identification information of the donor may instead or additionally be printed. Reading such information sets enables the processing circuitry 11 to acquire the identification information of the donor as the storage information.

Note that blood samples applicable to the present embodiment are not limited to those stored in a frozen state in a depository such as an umbilical blood bank. For example, a blood sample that has been collected from a donor and stored or transported in a non-frozen state may be used. The storage information may include the date of blood collection, identification information of the donor, etc.

After step S2, the processing circuitry 11 implements the estimating function 112 to estimate hematopoietic stem cell number information for the number of hematopoietic stem cells (step S3). This hematopoietic stem cell number information is one example of the tissue stem cell number information. In step S3, the processing circuitry 11 may estimate the hematopoietic stem cell number information using a trained model.

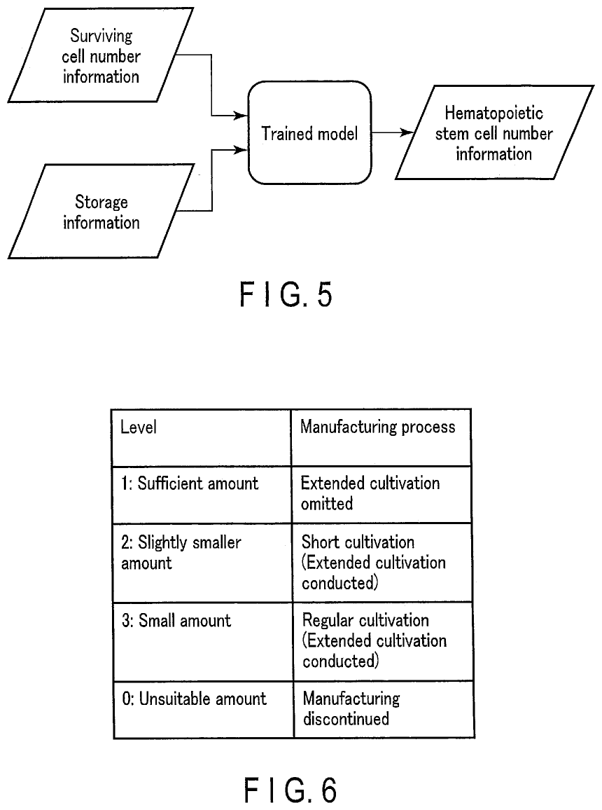

FIG. 5 is a diagram schematically showing input and output of a trained model that may be employed in step S3. As shown in FIG. 5, the trained model is a machine learning model with one or more parameters trained so that the hematopoietic stem cell number information is output using, as input, the surviving cell number information acquired in step S1 and the storage information acquired in step S2. Such a machine learning model may be a neural network, a support vector machine, etc. The output hematopoietic stem cell number information is constituted by, as one example, numerical information for the number or the ratio of hematopoietic stem cells. The number or the ratio of hematopoietic stem cells here is the number or the ratio of hematopoietic stem cells estimated to be included in the surviving cells at the time of measuring the to-be-input surviving cell number information. More specifically, it is the number or the ratio of hematopoietic stem cells included in the blood sample prior to the extended cultivation at the extended cultivator 33. The trained model is generated in advance by the training function 114 and stored in the memory 13.

The input and output of the trained model are not limited to the concrete examples discussed above. For example, as input, the surviving cell number information may be optical image information or optical spectrum information for the blood sample generated by the cell measuring apparatus 20 in step S1. Such optical image information or optical spectrum information are information indicative of the current conditions of the blood sample and are expected to show a correlation with the hematopoietic stem cell number information of the blood sample to some extent.

After step S3, the processing circuitry 11 implements the determining function 113 to determine a process for manufacturing induced pluripotent stem cells based on the hematopoietic stem cell number information estimated in step S3 (step S4). In step S4, the processing circuitry 11 determines the manufacturing process that corresponds to the hematopoietic stem cell number information estimated in step S3, based on the correspondence relationships between multiple levels for the hematopoietic stem cell number information and the respective, multiple manufacturing processes. Such correspondence relationships may be put into implementation using, for example, one or more look-up tables (LUTs). Such an LUT will be called a “manufacturing process table”. The manufacturing process table is generated in advance by the training function 114 and stored in the memory 13.

FIG. 6 is a diagram showing an example of the manufacturing process table used in step S4. As shown in FIG. 6, the manufacturing process table associates levels with manufacturing processes. The levels represent multiple degrees set according to values of the number or the ratio of hematopoietic stem cells. Each level is defined by a range of values of the number or the ratio of hematopoietic stem cells. The ranges of values of the number or the ratio of hematopoietic stem cells for the respective levels are, for example, determined in advance by the training function 114. The ranges may instead or additionally be set to any given ranges by an operator or the like via the input device 17, etc. The manufacturing processes each indicate a manufacturing process suitable for the corresponding level and to be adopted by the pluripotent stem cell manufacturing apparatus 30 to manufacture the induced pluripotent stem cells. The phrase “suitable for the corresponding level” here means, in more concrete terms, that the manufacturing process can manufacture a sufficient amount of induced pluripotent stem cells while consuming minimum possible time and cost for the manufacture. That is, the corresponding relationships between the levels and the manufacturing processes are determined so that the quality of induced pluripotent stem cells is well balanced with the time and costs required for their manufacture.

Level “1” is a level where the number or the ratio of pluripotent stem cells is found sufficient. The manufacturing process in the case of level “1” may omit the extended cultivation at the extended cultivator 33. This is because, as long as the number or the ratio of hematopoietic stem cells contained in the blood sample is sufficient, a sufficient amount of induced pluripotent stem cells will be manufactured without conducting the extended cultivation. It is possible to cut the time required for the extended cultivation as well as the expense incurred due to each reagent, by omitting the extended cultivation.

Level “2” is a level where the number or the ratio of pluripotent stem cells is found to be slightly smaller. The manufacturing process in the case of level “2” may include conducting the extended cultivation for a short time at the extended cultivator 33. As long as the number or the ratio of hematopoietic stem cells contained in the blood sample is only slightly smaller, conducting the extended cultivation for a short time will yield a sufficient amount of induced pluripotent stem cells at a reduced time for the extended cultivation and a reduced expense for reagents as compared to regular extended cultivation. Note that the time or duration of the extended cultivation conducted in this case may be discretionarily set to a time period shorter than the regular extended cultivation.

Level “3” is a level where the number or the ratio of pluripotent stem cells is found to be small. The manufacturing process in the case of level “3” may include conducting the extended cultivation for a regular time period at the extended cultivator 33. When the number or the ratio of hematopoietic stem cells contained in the blood sample is small, it is necessary to conduct the extended cultivation for a regular time period so that a sufficient amount of induced pluripotent stem cells is obtained. The regular time period adopted for the extended cultivation is generally about one week, but this may be adjusted to any time period according to blood samples, cultivation environment, and so on.

Level “0” is a level where the number or the ratio of pluripotent stem cells is significantly small and therefore not suitable for the manufacturing of induced pluripotent stem cells. The manufacturing process in the case of level “0” may discontinue the manufacturing of the induced pluripotent stem cells by the pluripotent stem cell manufacturing apparatus 30. This is because proceeding with the extended cultivation would not provide an increase of the hematopoietic stem cells, and the manufacturing of induced pluripotent stem cells will fail. Discontinuing the manufacturing can reduce the time and the expenses which are otherwise required by the process of manufacturing induced pluripotent stem cells and preparation of reagents, etc.

In step S4, the processing circuitry 11 specifies the level to which the hematopoietic stem cell number information estimated in step S3 belongs, from among the multiple levels defined in the manufacturing process table. The processing circuitry 11 then searches the manufacturing process table using this specified level as a search key, and determines the manufacturing process associated with the level.

Note that the description has assumed that the processing circuitry 11 in step S3 estimates the number or the ratio of hematopoietic stem cells as the hematopoietic stem cell number information, and in step S4 determines the level to which the number or the ratio of hematopoietic stem cells belongs, so as to determine the manufacturing process corresponding to the level. The level used here represents information according to the number or the ratio of hematopoietic stem cells, and as such, it is one example of the hematopoietic stem cell number information. That is, the processing circuitry 11 in step S3 may estimate the level by applying the surviving cell number information and the storage information to a trained model. For such a configuration, the trained model can be generated through training of one or more parameters so that when the surviving cell number information and the storage information are input to the model, the level according to this surviving cell number information is given as output.

The description has also assumed that the manufacturing processes set in the manufacturing process table are defined using conditions such as whether or not the extended cultivation should be conducted and/or how long the extended cultivation should be conducted. However, this is not a limitation. For example, each manufacturing process may be defined using manufacturing steps to be performed among the steps available in the manufacturing process. In this case, as one example, a manufacturing process without the extended cultivation may be defined to be a flow of unwanted substance removal→inducing factor introduction→pluripotent stem cell cultivation→storage.

After step S4, the processing circuitry 11 implements the display controlling function 115 to display the hematopoietic stem cell number information estimated in step S3 and the manufacturing process determined in step S4 (step S5). In step S5, the processing circuitry 11 causes the display 15 to display the hematopoietic stem cell number information and the manufacturing process in a predetermined layout.

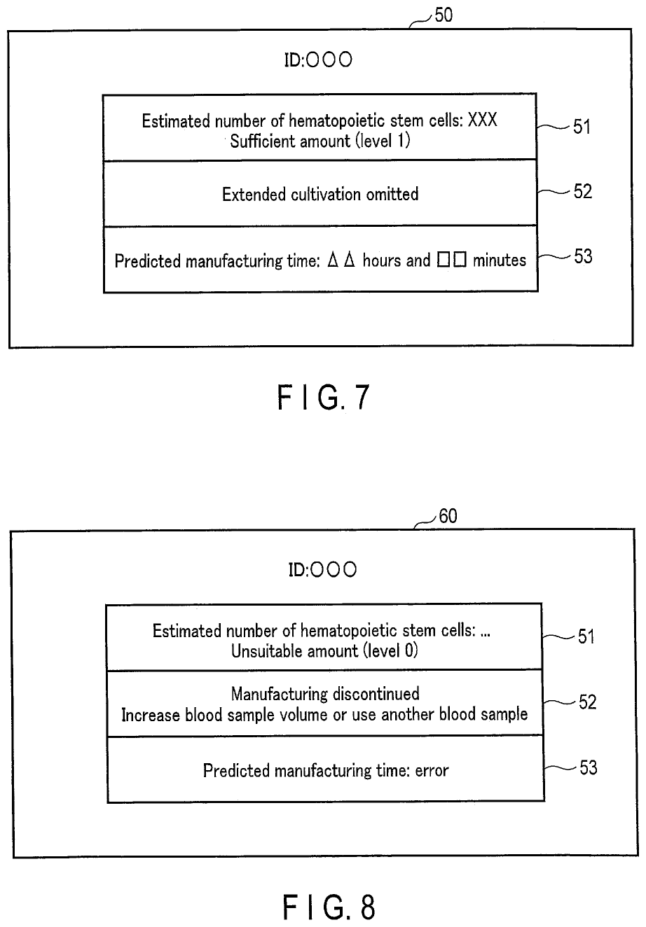

FIG. 7 is a diagram showing an example of a display screen 50 displaying, in step S5, the hematopoietic stem cell number information and the manufacturing process. The display screen 50 here is a presentation on the display 15. As shown in FIG. 7, the display screen 50 includes an indication of identification information ID of the donor or the blood sample. Also, the display screen 50 includes display regions 51, 52, and 53. The display region 51 is for displaying the hematopoietic stem cell number information estimated in step S3. The display region 51 may instead or additionally display the level for the hematopoietic stem cell number information estimated in step S3. The display region 52 is for displaying the manufacturing process determined in step S4. The display region 53 is for displaying a predicted manufacturing time for the manufacturing process determined in step S4. The predicted manufacturing time is calculated or determined by the processing circuitry 11 according to the manufacturing process determined in step S4. For example, a standard processing time for each step for manufacturing induced pluripotent stem cells is set, and the predicted manufacturing time can be calculated by summing up such standard processing times of the respective manufacturing steps to be performed. As another option, the manufacturing process table may associate the predicted manufacturing times with the respective levels.

In one example, when the manufacturing process corresponding to level “1” is determined in step S4, the display region 51 displays “Estimated number of hematopoietic stem cells: XXX” and “Sufficient amount (level 1)”, the display region 52 displays “Extended cultivation omitted”, and the display region 53 displays “Predicted manufacturing time: ΔΔ hours and □□ minutes”, as shown in FIG. 7. With the display screen 50, the operator or the like can confirm the hematopoietic stem cell number information, the manufacturing process, etc. in advance. The operator or the like can also know the predicted manufacturing time in advance.

FIG. 8 is a diagram showing another example of display presentation, namely, a display screen 60 displaying the hematopoietic stem cell number information and the manufacturing process. The display screen 60 is a presentation on the display 15. This display screen 60 is an example of display when the discontinuation of manufacturing corresponding to level “0” is determined in step S4. In this case, the display region 51 displays “Estimated number of hematopoietic stem cells: . . . ” and “Unsuitable amount (level 0)”, the display region 52 displays “Manufacturing discontinued” and “Increase blood sample volume or use another blood sample”, and the display region 53 displays “Predicted manufacturing time: error”. With the display screen 60, the operator or the like can confirm that the hematopoietic stem cell number is not suitable and the manufacturing will be discontinued. The operator or the like here may request that the umbilical blood depository, etc. resend the blood sample from the same donor in an increased amount. It is also possible to restart the process from step S1 for blood samples from other donors.

When the determination in step S4 is made based on level “1”, “2”, or “3”, the communication device 19 sends information for the manufacturing process determined in step S4 from the tissue stem cell number estimating apparatus 10 to the pluripotent stem cell manufacturing apparatus 30.

After step S5, the control circuitry 31 of the pluripotent stem cell manufacturing apparatus 30 manufactures the induced pluripotent stem cells having an origin in the donor of the blood sample, according to the manufacturing process determined in step S4 (step S6). In step S6, the control circuitry 31 controls the unwanted substance remover 32, the extended cultivator 33, the tissue stem cell extractor 34, the factor introducer 35, the pluripotent stem cell cultivator 36, the storage 37, the first switcher 38, and the second switcher 39, so that induced pluripotent stem cells are manufactured according to the manufacturing process determined in step S4.

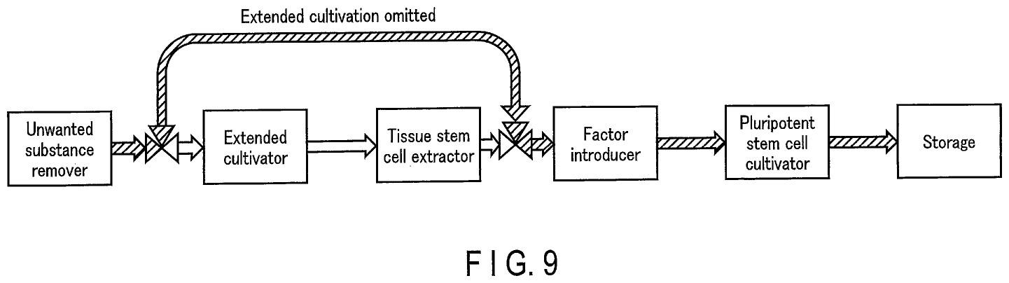

FIG. 9 is a diagram schematically showing a process for manufacturing induced pluripotent stem cells in the case of level “1” (extended cultivation omitted). As shown in FIG. 9, in the case of level “1”, the first switcher 38 is caused to open its inlet and second outlet while closing its first outlet, and the second switcher 39 is caused to open its second inlet and outlet while closing its first inlet. Accordingly, the flow channels R2, R3, and R4 which form a route from the unwanted substance remover 32 to the factor introducer 35 via the extended cultivator 33 and the tissue stem cell extractor 34 are closed, while the flow channel R8 for directly reaching the factor introducer 35 from the unwanted substance remover 32 is opened. Thus, in this case, the blood sample is first fed to the unwanted substance remover 32, where an unwanted substance or substances are removed from the blood sample. It has been estimated that the blood sample after the removal step contains a sufficient amount of hematopoietic stem cells. These hematopoietic stem cells are then guided via the flow channels R1, R8, and R5, and directly fed to the factor introducer 35, where the inducing factor is introduced into the hematopoietic stem cells. The introduction of the inducing factor causes the hematopoietic stem cells to be reprogrammed, and induced pluripotent stem cells are thereby established. The induced pluripotent stem cells are guided via the flow channel R6 and fed to the pluripotent stem cell cultivator 36, where the induced pluripotent stem cells are cultivated. Since the amount of the hematopoietic stem cells is sufficient, the manufacturing of a sufficient amount of induced pluripotent stem cells is expected. The induced pluripotent stem cells are then fed to the storage 37 via the flow channel R7 and sealed in one or more containers for storage.

When the amount of hematopoietic stem cells contained in the blood sample is estimated to be sufficient, the extended cultivation is omitted as shown in FIG. 9. In this manner, the time required for the extended cultivation and also the expenses necessary for various reagents, etc. can be cut while providing a sufficient amount of induced pluripotent stem cells.

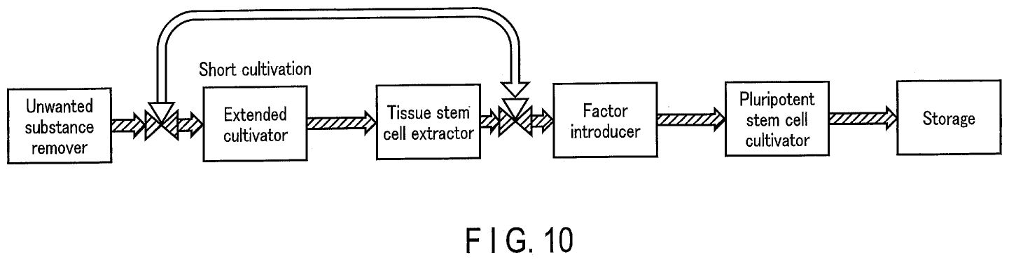

FIG. 10 is a diagram schematically showing a process for manufacturing induced pluripotent stem cells in the case of level “2” (short cultivation). As shown in FIG. 10, in the case of level “2”, the first switcher 38 is caused to open its inlet and first outlet while closing its second outlet, and the second switcher 39 is caused to open its first inlet and outlet while closing its second inlet. Accordingly, the flow channels R2, R3, and R4 which form a route from the unwanted substance remover 32 to the factor introducer 35 via the extended cultivator 33 and the tissue stem cell extractor 34 are opened, while the flow channel R8 for directly reaching the factor introducer 35 from the unwanted substance remover 32 is closed. It has been estimated that the blood sample after the removal of unwanted substances by the unwanted substance remover 32 contains a slightly smaller amount of hematopoietic stem cells. The hematopoietic stem cells are subjected to extended cultivation for a short time at the extended cultivator 33 and then extracted by the tissue stem cell extractor 34. The extracted hematopoietic stem cells are fed to the factor introducer 35. Subsequently, induced pluripotent stem cells are established by the factor introducer 35, and these induced pluripotent stem cells are cultivated at the pluripotent stem cell cultivator 36. The induced pluripotent stem cells are then sealed in one or more containers and stored at the storage 37.

When the amount of hematopoietic stem cells contained in the blood sample is estimated to be slightly smaller, the extended cultivation is only conducted for a short time as indicated in FIG. 10. In this manner, despite the hematopoietic stem cells being provided in a slightly smaller amount, the time required for the extended cultivation and also the expense necessary for various reagents, etc. can be reduced while providing a sufficient amount of induced pluripotent stem cells.

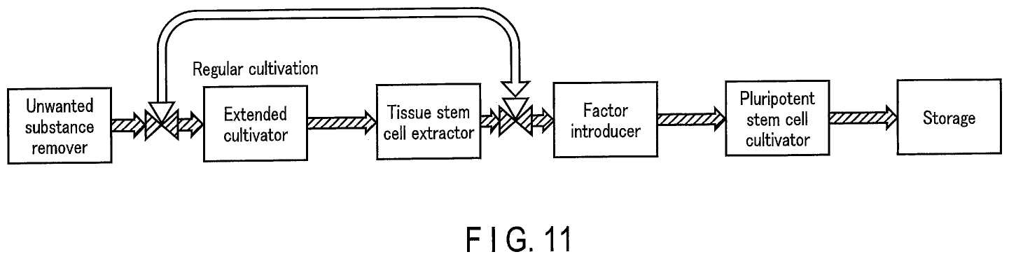

FIG. 11 is a diagram schematically showing a process for manufacturing induced pluripotent stem cells in the case of level “3” (regular cultivation). As shown in FIG. 11, in the case of level “3”, the first switcher 38 is caused to open its inlet and first outlet while closing its second outlet, and the second switcher 39 is caused to open its first inlet and outlet while closing its second inlet, as in the case of level “2”. Accordingly, the flow channels R2, R3, and R4 are opened, while the flow channel R8 is closed. It has been estimated that the blood sample after the removal of unwanted substances by the unwanted substance remover 32 contains a small amount of hematopoietic stem cells. The hematopoietic stem cells are subjected to extended cultivation for a regular time period at the extended cultivator 33 and then extracted by the tissue stem cell extractor 34. The extracted hematopoietic stem cells are fed to the factor introducer 35. Subsequently, induced pluripotent stem cells are established by the factor introducer 35, and these induced pluripotent stem cells are cultivated at the pluripotent stem cell cultivator 36. The induced pluripotent stem cells are then sealed in one or more containers and stored at the storage 37.

When the amount of hematopoietic stem cells contained in the blood sample is estimated to be a small amount, the extended cultivation is conducted for a regular time period, as indicated in FIG. 11. In this manner, despite the amount of hematopoietic stem cells being small, a sufficient amount of induced pluripotent stem cells can be provided.

Upon performing step S6, the operation of manufacturing induced pluripotent stem cells by the pluripotent stem cell manufacturing system 1 according to the embodiment comes to an end.

Note that the manufacturing operation for induced pluripotent stem cells is not limited to what is shown in FIG. 4, but may include various modifications. For example, step S1 and step S2 may be transposed. In some instances, step S5 may be omitted. Also, it is not required in step S5 to always display both the hematopoietic stem cell number information and the storage information, but only one of the hematopoietic stem cell number information and the storage information may be displayed.

The description has assumed four levels for the hematopoietic stem cell number information, but the number of levels is not limited. For example, level “0” may be omitted. The purpose may be served by setting at least two levels, namely, the first level representing an unsuitable number of hematopoietic stem cells and the second level representing an appropriate number of hematopoietic stem cells. The first level here may be associated with a manufacturing process in which extended cultivation for a given time period is conducted, and the second level may be associated with a manufacturing process without the extended cultivation.

Levels “2” and “3” may each be divided into multiple portions according to cultivation times. For example, level “2” may be divided into multiple sub-levels corresponding to respective cultivation times shorter than the regular time period. Level “3” may be divided into multiple sub-levels corresponding to respective cultivation times longer than the regular time period. Subdividing the levels in this manner can secure the options for more appropriate cultivation times according to the conditions of blood samples, and therefore, enables more accurate and finer adjustment of the quality of induced pluripotent stem cells, as well as their manufacturing time and costs, etc.

The description has assumed that the manufacturing process determined in step S4 is automatically carried out in the manufacturing operation discussed above, but the operation is not limited to such a form. In another example, the determined manufacturing process may be withheld until approval from the operator or the like.

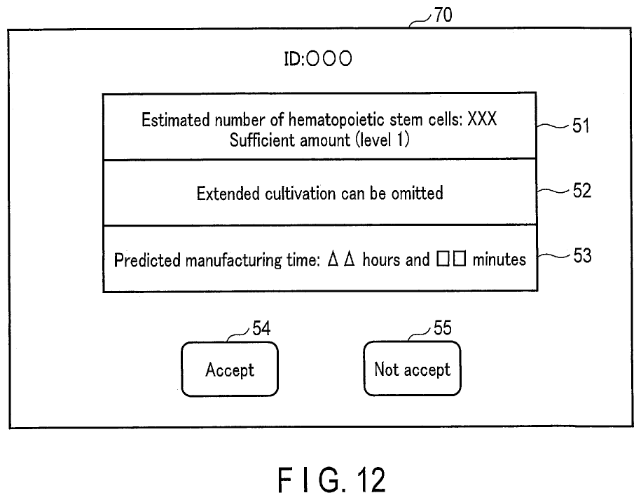

FIG. 12 is a diagram showing another example of display presentation, namely, a display screen 70 presented in step S5. The display screen 70 includes an accept button 54 and a non-accept button 55, in addition to the display regions 51 to 53. The accept button 54 is a graphical user interface (GUI) button for informing the processing circuitry 11 that the manufacturing process determined in step S4, that is, the manufacturing process displayed in the display region 52, is to be adopted. The non-accept button 55 is a GUI button for informing the processing circuitry 11 that the manufacturing process determined in step S4, that is, the manufacturing process displayed in the display region 52, is not to be adopted.

When the accept button 54 is pressed using the input device 17, the processing circuitry 11 conveys the manufacturing process determined in step S4 to the pluripotent stem cell manufacturing apparatus 30. Accordingly, induced pluripotent stem cells are manufactured according to this manufacturing process. When the non-accept button 55 is pressed using the input device 17, the processing circuitry 11 discards the manufacturing process determined in step S4 and conveys a predetermined normal manufacturing process to the pluripotent stem cell manufacturing apparatus 30. For example, the manufacturing process that conducts extended cultivation for a regular time period may be set as such as a normal manufacturing process. Induced pluripotent stem cells are therefore manufactured with the normal manufacturing process. This configuration of prompting approval of the operator or the like enables the manufacturing of induced pluripotent stem cells to proceed with the manufacturing process intended by the operator, etc.

Next, generation of the trained model and the manufacturing process table will be described.

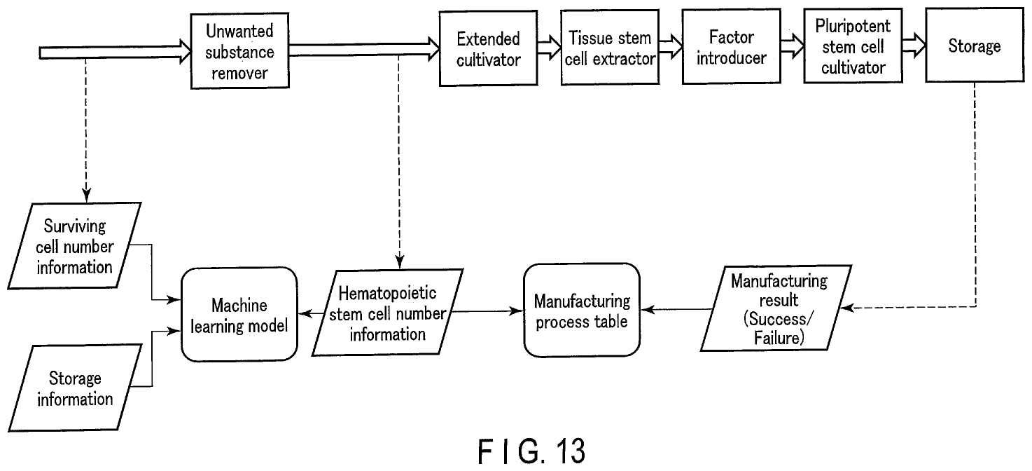

FIG. 13 is a diagram schematically showing a training process for the trained model and the manufacturing process table. As shown in FIG. 13, the training process includes collection of the surviving cell number information, the storage information, the hematopoietic stem cell number information, and also information on the result of manufacturing (“manufacturing result information”). The set of the surviving cell number information, the storage information, and the hematopoietic stem cell number information is called a “training sample” since this set is used for training the trained model. The training sample is collected in the course of manufacturing induced pluripotent stem cells from the blood sample through the extended cultivation. The surviving cell number information and the storage information are used as input data among the training sample. The hematopoietic stem cell number information is used as ground truth data or output data among the training sample. The hematopoietic stem cell number information and the manufacturing result information are used for generating the manufacturing process table.

As shown in FIG. 13, the processing circuitry 11 implements the acquiring function 111 to acquire the storage information of the blood sample from, for example, a blood data sheet or the like affixed to the blood sample container. The cell measuring apparatus 20 measures the blood sample before removal of unwanted substances by the unwanted substance remover 32, and acquires the surviving cell number information for the number of surviving cells contained in the blood sample. The surviving cell number information is acquired by the tissue stem cell number estimating apparatus 10 with the processing circuitry 11 implementing the acquiring function 111. Subsequently, one or more unwanted substances contained in the blood sample are removed by the unwanted substance remover 32. The cell measuring apparatus 20 then measures the blood sample that has undergone the removal of unwanted substances, and acquires the surviving cell number information for the number of surviving cells contained in this blood sample. Here, the surviving cell number information is acquired as the hematopoietic stem cell number information constituting the training sample, for the number of hematopoietic stem cells in the surviving cells.

After that, the extended cultivation for a regular time period at the extended cultivator 33, the extraction of hematopoietic stem cells at the tissue stem cell extractor 34, the introduction of an inducing factor at the factor introducer 35, the cultivation of induced pluripotent stem cells at the pluripotent stem cell cultivator 36, and the storage of the induced pluripotent stem cells at the storage 37 are conducted. The induced pluripotent stem cells stored at the storage 37 are measured so that the manufacturing result information is obtained. The manufacturing result information includes an evaluation based on the number of the induced pluripotent stem cells as to whether the manufacturing has succeeded or failed. In one example, the number of the induced pluripotent stem cells stored at the storage 37 is measured by the cell measuring apparatus 20 and compared to a threshold. If the number is larger than the threshold, the manufacturing is evaluated to be a success, and if the number is smaller than the threshold, the manufacturing is evaluated to be a failure. The information on the evaluation is acquired by the tissue stem cell number estimating apparatus 10 with the processing circuitry 11 implementing the acquiring function 111. In this manner, for each of various blood samples, the surviving cell number information, the storage information, the hematopoietic stem cell number information, and the manufacturing result information are collected.

Upon collecting the surviving cell number information, the storage information, the hematopoietic stem cell number information, and the manufacturing result information, the training process for a machine learning model, as well as the training of the manufacturing process table are conducted. First, training of a machine learning model will be explained. The processing circuitry 11 implements the training function 114 to train a machine learning model based on multiple training samples to generate a trained model adapted to receive, as input, the surviving cell number information and the storage information and output the hematopoietic stem cell number information. Various combinations of the surviving cell number information and the hematopoietic stem cell number information are possible. For example, the generated trained model may use, as input, the number of surviving cells together with the storage information to return the number of the hematopoietic stem cells as output. In another instance, the generated trained model may use, as input, the ratio of surviving cells together with the storage information to return the ratio of the hematopoietic stem cells as output. In yet another instance, the generated trained model may use, as input, the image information and/or the optical spectrum information of surviving cells together with the storage information to return the number or the ratio of the hematopoietic stem cells as output.

The hematopoietic stem cell number information for the training may be information after the extended cultivation. For such instances, for example, the number of hematopoietic stem cells after the extended cultivation at the extended cultivator 33 is measured by the cell measuring apparatus 20, the flow cytometer device, or the like. The processing circuitry 11 calculates the number of hematopoietic stem cells before the extended cultivation by multiplying the measured number of hematopoietic stem cells after the extended cultivation by a coefficient. This coefficient may be determined in an empirical manner according to the extended cultivation time, the cultivation conditions, etc. The thus-calculated number of hematopoietic stem cells before the extended cultivation may be used as the ground truth data in the training sample.

Next, training of the manufacturing process table will be explained. The processing circuitry 11 determines, according to one or more commands from an operator or the like via the input device 17, the number of level divisions (“level number”) for the hematopoietic stem cell number, the ranges (“level ranges”) of the hematopoietic stem cell number that delimits the respective levels, and the manufacturing processes corresponding to the respective levels. Here, the surviving cell number information, the hematopoietic stem cell number information, and the manufacturing result information may be analyzed so as to determine the level number and the level ranges. For example, the processing circuitry 11 may iteratively update or correct, starting from the level number and the level ranges that were tentatively determined, the level number and the level ranges using, as feedback, the hematopoietic stem cell number information and the manufacturing result information that are being iteratively acquired. The analyzing technique here may be statistical operations, machine learning processes, etc.

Note that, as mentioned above, the hematopoietic stem cell number information may be the levels for the number of hematopoietic stem cells. For such instances, the processing circuitry 11 may determine the levels corresponding to the numbers of hematopoietic stem cells before the extended cultivation, and use the determined levels as the ground truth data in the training sample.

### Modification 1

According to Modification 1, the trained model may output a manufacturing process. Since the level and the manufacturing process is in one-to-one correspondence, it is also possible for the trained model to output the suitable manufacturing process. The trained model according to Modification 1 may be generated through the training process so that the trained model is adapted to use, as input, the surviving cell number information and the storage information to output the manufacturing process. The manufacturing processes as the ground truth data in the training sample may be determined by the processing circuitry 11 based on, for example, the number of hematopoietic stem cells measured using the blood sample after the removal of unwanted substances. More specifically, the level is determined based on the number of hematopoietic stem cells, and the manufacturing process corresponding to this level is adopted.

### Modification 2

The description of the foregoing embodiment has assumed that the pluripotent stem cell manufacturing apparatus 30 with the control circuitry 31 automatically operates the unwanted substance remover 32, the extended cultivator 33, the tissue stem cell extractor 34, the factor introducer 35, the pluripotent stem cell cultivator 36, and the storage 37. According to Modification 2, the unwanted substance remover 32, the extended cultivator 33, the tissue stem cell extractor 34, the factor introducer 35, the pluripotent stem cell cultivator 36, and the storage 37 may be manually operated by hand, etc. Modification 2, for example, makes it unnecessary to determine a manufacturing process in step S4 as shown in FIG. 4. Thus, in this modification, the processing circuitry 11 in step S5 causes the display of the tissue stem cell number information estimated in step S3. The operator, etc. may confirm the displayed tissue stem cell number information and then carry out the manufacturing of pluripotent stem cells according to the manufacturing process corresponding to this tissue stem cell number information.

More specifically, and for example, when the tissue stem cell number information corresponding to level “1” is displayed in step S5, the operator, etc. may operate the factor introducer 35 to introduce an inducing factor into the hematopoietic stem cells after the removal of unwanted substances at the unwanted substance remover 32, and operate the pluripotent stem cell cultivator 36 to cultivate the pluripotent stem cells. When the tissue stem cell number information corresponding to level “2” is displayed in step S5, the operator, etc. may operate the extended cultivator 33 to subject the hematopoietic stem cells to the extended cultivation for a short time after the removal of unwanted substances at the unwanted substance remover 32. Subsequent to this extended cultivation, the operator, etc. may extract the hematopoietic stem cells using the tissue stem cell extractor 34, operate the factor introducer 35 to introduce an inducing factor into the hematopoietic stem cells, and operate the pluripotent stem cell cultivator 36 to cultivate the pluripotent stem cells. When the tissue stem cell number information corresponding to level “3” is displayed in step S5, the operator, etc. may operate the extended cultivator 33 to subject the hematopoietic stem cells to the extended cultivation for a regular time period after the removal of unwanted substances at the unwanted substance remover 32. Subsequent to this extended cultivation, the operator, etc. may extract the hematopoietic stem cells using the tissue stem cell extractor 34, operate the factor introducer 35 to introduce an inducing factor into the hematopoietic stem cells, and operate the pluripotent stem cell cultivator 36 to cultivate the pluripotent stem cells.

Note that Modification 2 may also include the determination of a manufacturing process in step S4. In such instances, the processing circuitry 11 in step S5 causes the display of the manufacturing process determined in step S4. The operator, etc. may carry out the manufacturing of pluripotent stem cells according to the displayed manufacturing process.

### Modification 3

Configurations illustrated in FIGS. 1 to 3 are examples and the embodiments are not limited to them. In one example, the whole or a part of the tissue stem cell number estimating apparatus 10 may be incorporated into the pluripotent stem cell manufacturing apparatus 30. Examples of such partial incorporation include incorporation of the determining function 113 of the processing circuitry 11 into the pluripotent stem cell manufacturing apparatus 30. Also, the cell measuring apparatus 20 may be incorporated into the tissue stem cell number estimating apparatus 10 or the pluripotent stem cell manufacturing apparatus 30.

(In Sum)

According to the foregoing embodiments, the pluripotent stem cell manufacturing system 1 includes the tissue stem cell number estimating apparatus 10. The tissue stem cell number estimating apparatus 10 acquires the storage information for the storage of a sample from a donor, and the surviving cell number information for the number of surviving cells contained in the sample. The tissue stem cell number estimating apparatus 10 estimates the tissue stem cell number information for the number of tissue stem cells contained in the sample, based on the storage information and the surviving cell number information.

The configurations described above allow for the obtaining of the information on the number of tissue stem cells contained in a sample, which reflects the current conditions of the sample. Accordingly, a suitable manufacturing process that realizes a balance between the yields of the pluripotent stem cells and the manufacturing costs can be estimated from this tissue stem cell number information, and the operator, etc. can conduct the manufacturing of pluripotent stem cells according to this manufacturing process realizing a good balance. According to the embodiments, it is also possible to automatically determine the manufacturing process for pluripotent stem cells from the tissue stem cell number information, and therefore, pluripotent stem cells can be manufactured more easily according to the manufacturing process that realizes a good balance. Moreover, since the embodiments enable automation of the manufacturing of pluripotent stem cells, pluripotent stem cells can be manufactured more easily and stably according to the manufacturing process that realizes a good balance.

With at least one of the embodiments described above, pluripotent stem cells can be efficiently manufactured according to the conditions of samples.

The term “processor” used herein refers to, for example, a CPU or a GPU, or various types of circuitry, such as an application-specific integrated circuit (ASIC), a programmable logic device (e.g., a simple programmable logic device (SPLD), a complex programmable logic device (CPLD), or a field programmable gate array (FPGA)), and so on. The processor reads programs stored in storage circuitry and executes them to realize the intended functions. The programs may be incorporated directly in circuits of the processor, instead of being stored in the storage circuitry. According to such architecture, the processor reads the programs incorporated in its circuits and executes them to realize the functions. As another option, functions corresponding to the programs may be realized by a combination of logic circuits, instead of having the programs executed. The embodiments, etc., described herein do not limit each processor to a single circuitry-type processor. Multiple independent circuits may be combined and integrated as one processor to realize the intended functions. Furthermore, multiple components or features as given in FIG. 1, FIG. 2, and FIG. 3 may be integrated as one processor to realize their functions.

While certain embodiments have been described, they have been presented by way of example only, and they are not intended to limit the scope of the inventions. These embodiments may be worked in a variety of other forms with various omissions, substitutions, changes, and combinations without departing from the spirit of the inventions. The embodiments and their modifications are covered by the accompanying claims and their equivalents, as would fall within the scope and the gist of the claimed inventions.

1. A pluripotent stem cell manufacturing system comprising
processing circuitry configured to
acquire storage information for storage of a sample from a donor, and surviving cell number information for a number of surviving cells contained in the sample, and
estimate tissue stem cell number information for a number of tissue stem cells contained in the sample, based on the storage information and the surviving cell number information.

2. The pluripotent stem cell manufacturing system according to claim 1, wherein the processing circuitry is configured to determine, based on the tissue stem cell number information, a manufacturing process for manufacturing pluripotent stem cells from the tissue stem cells.

3. The pluripotent stem cell manufacturing system according to claim 2, further comprising a manufacturing apparatus configured to manufacture the pluripotent stem cells from the tissue stem cells according to the manufacturing process, the pluripotent stem cells having an origin in the donor.

4. The pluripotent stem cell manufacturing system according to claim 2, wherein the processing circuitry is configured to
determine, according to the tissue stem cell number information, a level for the number of the tissue stem cells contained in the sample, and
determine, based on the determined level and a correspondence between a plurality of predetermined levels and a plurality of manufacturing processes, the manufacturing process corresponding to the determined level.

5. The pluripotent stem cell manufacturing system according to claim 4, wherein the processing circuitry is configured to
adopt a manufacturing process omitting extended cultivation of the tissue stem cells if the determined level is a first level, and
adopt a manufacturing process including the extended cultivation if the determined level is a level other than the first level.

6. The pluripotent stem cell manufacturing system according to claim 4, wherein the processing circuitry is configured to
adopt a manufacturing process omitting extended cultivation of the tissue stem cells if the determined level is a first level,
adopt a manufacturing process including the extended cultivation for a first time if the determined level is a second level lower than the first level, and
adopt a manufacturing process including the extended cultivation for a second time longer than the first time if the determined level is a third level lower than the second level.

7. The pluripotent stem cell manufacturing system according to claim 4, wherein the processing circuitry is configured to determine discontinuation of manufacturing of the pluripotent stem cells if the determined level is a fourth level unsuitable for the manufacturing of the pluripotent stem cells.

8. The pluripotent stem cell manufacturing system according to claim 1, wherein
the sample comprises blood of the donor, and
the tissue stem cells are hematopoietic stem cells.

9. The pluripotent stem cell manufacturing system according to claim 8, wherein the storage information comprises information indicative of a number of CD34 positive cells at an initial time point for storage of the sample, a ratio of the CD34 positive cells at the initial time point, a number of storage years, and/or a storage period.

10. The pluripotent stem cell manufacturing system according to claim 9, wherein the storage information further comprises identification information of the donor.

11. The pluripotent stem cell manufacturing system according to claim 8, wherein the sample comprises umbilical blood which has been stored in an umbilical blood depository.

12. The pluripotent stem cell manufacturing system according to claim 1, wherein the surviving cell number information comprises numerical information for the number of the surviving cells contained in the sample, numerical information for a ratio of the surviving cells, image information for the sample, and/or optical spectrum information for the sample.

13. The pluripotent stem cell manufacturing system according to claim 1, wherein the processing circuitry is configured to estimate the tissue stem cell number information for the number of the tissue stem cells estimated to be contained in the sample before extended cultivation.

14. The pluripotent stem cell manufacturing system according to claim 1, wherein
the processing circuitry is configured to estimate the tissue stem cell number information by applying the storage information and the surviving cell number information to a trained model,
the trained model being a machine learning model trained to input the storage information and the surviving cell number information and output the tissue stem cell number information.

15. The pluripotent stem cell manufacturing system according to claim 1, further comprising a display configured to display the tissue stem cell number information.

16. The pluripotent stem cell manufacturing system according to claim 2, further comprising a display configured to display the manufacturing process.

17. A pluripotent stem cell manufacturing system comprising
processing circuitry configured to
acquire a plurality of training samples corresponding to a plurality of donor samples, respectively, wherein the plurality of training samples each comprises storage information for storage of the corresponding donor sample, surviving cell number information for a number of surviving cells contained in the corresponding donor sample, and tissue stem cell number information for a number of tissue stem cells contained in the corresponding donor sample, and
train a machine learning model based on the plurality of training samples to generate a trained model that inputs the storage information and the surviving cell number information and outputs the tissue stem cell number information.
